(19) United States
(12) Patent Application Publication
(10) Pub. No.: US 2025/0239350 A1
(43) Pub. Date: Jul. 24, 2025

(54) INSULIN DOSAGE DETERMINATION SYSTEM BASED ON PERSONALIZED ARTIFICIAL INTELLIGENCE

(71) Applicants: CureStream Co.,Ltd, Daegu (KR); POSTECH Research and Business Development Foundation, Gyeongsangbuk-do (KR)

(72) Inventors: Sung Min Park, Gyeongsangbuk-do (KR); Vega Pradana Rachim, Gyeongsangbuk-do (KR)

(21) Appl. No.: 19/010,322

(22) Filed: Jan. 6, 2025

(30) Foreign Application Priority Data
Jan. 19, 2024 (KR) 10-2024-0008902
Dec. 3, 2024 (KR) 10-2024-0177019

(51) Int. Cl. G16H 20/17; A61M 5/142

(52) U.S. Cl. CPC G16H 20/17; A61M 5/142; A61M 2005/14292; A61M 2202/0007; A61M 2202/04; A61M 2205/10; A61M 2205/3303; A61M 2205/50; A61M 2230/005; A61M 2230/201

(57) ABSTRACT

Provided is a personalized artificial intelligence-based insulin dose determination system includes an interface layer capable of communicating with the insulin pump and the continuous blood glucose system, and signal-processing information from the insulin pump and the continuous blood glucose system, a control layer receiving the signal-processed information from the interface layer and generating output information associated with an insulin infusion amount, an outer safety layer determining whether or not the output information generated from the control layer satisfies a preset threshold condition, and delivering the output information to the interface layer when the output information satisfies the preset threshold condition, and a personalized safety layer receiving prescription information including Total Daily Dose of Insulin (TDD) information of the user from the outside to determine a personalized safety control variable, and transmitting the determined control variable as an input variable of the control layer.

### CROSS-REFERENCE TO RELATED APPLICATION

This application claims priority of Korean Patent Application No. 10-2024-0008902, filed on Jan. 19, 2024, and priority of Korean Patent Application No. 10-2024-0177019, filed on Dec. 3, 2024, in the KIPO (Korean Intellectual Property Office), the disclosure of which is incorporated herein entirely by reference.

### BACKGROUND OF THE INVENTION

### Field of the Invention

The present invention relates to a personalized artificial intelligence-based insulin dose determination system.

### Description of the Related Art

Diabetes can be characterized by hyperglycemia and relative insulin deficiencies. There are two main types of diabetes, type 1 diabetes (insulin dependent diabetes mellitus) and type 2 diabetes (non-insulin dependent diabetes mellitus). In some cases, diabetes is also characterized by insulin resistance.

For the treatment of such diabetes, various non-invasive transdermal (e.g., transdermal) and/or insertable electrochemical sensors have been developed to continuously detect and/or quantify blood glucose values. Continuous glucose monitoring is becoming more popular as a way to easily monitor glucose levels.

Along with the continuous glucose monitoring, a bolus calculator device is used, and in this case, variables such as basal insulin, a Correction Factor (CF), a carbohydrate-to-insulin ratio (CIR), and the like, have to be measured and input.

### SUMMARY OF THE INVENTION

Accordingly, an object to be achieved by the present disclosure is to provide a system capable of more effectively determining variables used in a precision insulin control system, and a bolus calculator using the same.

In order to achieve the above object, the present invention provides a personalized artificial intelligence-based insulin dose determination system by a computing terminal capable of communicating with an insulin pump and a continuous blood glucose system used by a user, the system comprising: an interface layer capable of communicating with the insulin pump and the continuous blood glucose system, and signal-processing information from the insulin pump and the continuous blood glucose system; a control layer receiving the signal-processed information from the interface layer, and generating output information related to an insulin injection amount; an outer safety layer determining whether the output information generated from the control layer satisfies a preset threshold condition, and transmitting the output information to the interface layer when the output information satisfies the preset threshold condition; and a personalized safety layer receiving prescription information including Total Daily Dose of Insulin (TDD) information of the user from the outside, determining a personalized safety control variable, and transmitting the determined control variable as an input variable of the control layer.

In one embodiment of the present invention, the control layer determines, as output information, the amount of insulin injected into the user using at least one of a continuous blood glucose level (g), an insulin-on-board (iob), a continuous blood glucose level (dg/dt or vg), and a continuous blood glucose acceleration (d2g/dt2 or ag) measured by the continuous blood glucose system as input information from the signal-processed information.

In one embodiment of the present invention, iobt at time t is determined by the following equation (1).

$$i o {b}_{t} = {\sum }_{k = 0}^{n - 1} {i}_{t - k} \cdot ( 1 \to {F}_{k} ( S F ) ) \qquad ( 1 )$$

where iₜ₋ₖ is the insulin dose injected in the k time step before t, and Fₖ is the gamma cumulative density function (CDF) using SF as a scaling factor.

In an embodiment of the present disclosure, the control layer determines the amount of insulin injected into the user as output information using a deep-reinforced learning model.

In an embodiment of the present invention, the deep-reinforced learning model uses a soft actor critical (SAC) algorithm, the control layer determines a blood glucose control policy of the soft actor critical (SAC) algorithm at predetermined time intervals, and the control layer trains the soft actor critical (SAC) algorithm model which is trained for the purpose of maximizing an objective function according to the following equation.

The control layer uses the SAC (Soft Actor Critical) algorithm model for the purpose of maximizing the objective function according to the following equation (4).

$$J ( π ) = {\sum }_{t = 0}^{T} {E}_{( {s}_{t} , {a}_{t} ) \sim P ( {s}_{t - 1} , {π}_{ϕ} ( {s}_{t - 1} ) )} [ R ( {s}_{t} , {a}_{t} ) + α H ( {π}_{ϕ} ( . \vert {s}_{t} ) ) ] \qquad ( 4 )$$

where ΣₜE(sₜ,aₜ)˜P(sₜ₋₁,π_{ϕ}(sₜ₋₁))[R(sₜ,aₜ)] is the compensation sum, H is the entropy, and α is the temperature parameter.

In an embodiment of the present invention, the control layer determines the insulin injection amount as an output variable within any one or more of the following three limit value ranges:
Insulin infusion during the day (SD)
Insulin infusion during the night (SN)
Maximum insulin-on-board value (iobmax).

In one embodiment of the present invention, the insulin injection amount is determined by the following equation.

$$\overline{i} := \{ \begin{matrix}\overline{i} . {S}_{N} + π , & {τ}_{1} < {t}_{i} \leq {τ}_{2} \\ \overline{i} . {S}_{D} + π , & otherwise\end{matrix}$$

where ī is information on the output of a control layer, t₁ is time during the day, τ₁ and τ₂ are start time and end time, and π is BRₘᵢₙ/2, BRmin is BR/60 [U/min], and BR is basal insulin rate.

In an embodiment of the present disclosure, the insulin injection amount SN during the night is a value obtained by multiplying the insulin injection amount SD during the day by a coefficient between 0 and 1.

In one embodiment of the present invention, the coefficient is determined according to TDD (Total Daily Dose of Insulin).

In an embodiment of the present disclosure, the maximum insulin-on-board value (iobmax) is determined as a constant according to the TDD (Total Daily Dose of Insulin), and the maximum insulin-on-board value (iobmax) is provided as a threshold of the outer safety layer.

It provides an accurate insulin management (AIM) system applicable in large-capacity endocrine clinicians and shows an excellent balance between high accuracy and characteristics that are short, easy to remember and easy to use to reduce administration errors.

### BRIEF DESCRIPTION OF THE DRAWINGS

The above and other features and advantages will become more apparent to those of ordinary skill in the art by describing in detail exemplary embodiments with reference to the attached drawings, in which:

FIG. 1 is a block diagram of a completely closed-loop insulin dose determination system architecture according to an embodiment of the present invention.

FIG. 2 is an operational schematic diagram of a system having a layered structure according to an embodiment of the present invention.

FIG. 3 is a conceptual diagram according to an embodiment of the present invention.

FIG. 4 is a diagram comparing generalized iob effects between two subjects with different TDD values.

FIG. 5 is a diagram illustrating a calibration iteration procedure according to an exemplary embodiment of the present invention.

FIG. 6 is a graph illustrating a correlation between TDD and a control variable SD.

FIG. 7 is a graph comparing three CHOs having average contents of 40 g, 80 g, and 60 g for 1 day, and shows a result of comparing (a) an open loop (including a meal guide) using a conventional basal Bolus calculator with (b) a completely closed loop (without a meal guide) according to the present invention.

FIGS. 8A to 8C are the result of using the first two weeks of data obtained in the entered meal-open loop treatment and the last four weeks of data obtained in the fully closed loop of the present invention with unentered meal-personal AI.

In the following description, the same or similar elements are labeled with the same or similar reference numbers.

### DETAILED DESCRIPTION

The present invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes”, “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. In addition, a term such as a “unit”, a “module”, a “block” or like, when used in the specification, represents a unit that processes at least one function or operation, and the unit or the like may be implemented by hardware or software or a combination of hardware and software.

Reference herein to a layer formed “on” a substrate or other layer refers to a layer formed directly on top of the substrate or other layer or to an intermediate layer or intermediate layers formed on the substrate or other layer. It will also be understood by those skilled in the art that structures or shapes that are “adjacent” to other structures or shapes may have portions that overlap or are disposed below the adjacent features.

In this specification, the relative terms, such as “below”, “above”, “upper”, “lower”, “horizontal”, and “vertical”, may be used to describe the relationship of one component, layer, or region to another component, layer, or region, as shown in the accompanying drawings. It is to be understood that these terms are intended to encompass not only the directions indicated in the figures, but also the other directions of the elements.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Preferred embodiments will now be described more fully hereinafter with reference to the accompanying drawings. However, they may be embodied in different forms and should not be construed as limited to the embodiments set forth herein. Rather, these embodiments are provided so that this disclosure will be thorough and complete, and will fully convey the scope of the disclosure to those skilled in the art.

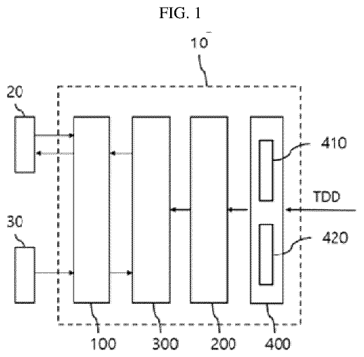

FIG. 1 is a block diagram of a completely closed-loop insulin dose determination system architecture according to an embodiment of the present invention. Hereinafter, the term “layer” refers to functionally separated software and/or hardware forming a layered architecture.

The system according to the present invention may be implemented in a terminal (e.g., a mobile phone, a tablet PC, etc.) that can communicate with an insulin pump and a continuous blood glucose system that are attached to a body used by a user and can be computed by itself, and the following layers may be configured to be divided in software in the terminal.

That is, the system according to the present invention is a device that is mounted on a terminal or the like and can be processed by a processor of the terminal, and the system 10 according to an embodiment of the present invention includes an interface layer 100 that can communicate with an insulin pump 20 and a continuous blood glucose meter 30 used by a user, and can receive information from the insulin pump 20 and the continuous blood glucose meter 30 and process a signal. In an embodiment of the present invention, the interface layer 100 may transmit information on the insulin injection amount determined by the system to the insulin pump 20 so that the insulin pump 20 may operate as much as the determined injection amount.

The system 10 includes: a control layer 200 configured to receive signal-processed information from the interface layer 100 and generate output information associated with the insulin injection amount; an outer layer 300 configured to determine whether the output information (i.e., the calculated insulin injection amount) generated from the control layer 200 satisfies a preset threshold condition, and transmit the output information to the interface layer when the output information satisfies the preset threshold condition; and a personalized safety layer 400 configured to receive prescription information including TDD (Total Daily Dose of Insulin) information of a user from the outside, determine a personalized safety control variable, and transmit the determined control variable as an input variable of the control layer.

In particular, the system according to an exemplary embodiment of the present invention includes the external safety unit 200 between the interface unit 100 for receiving a signal by communicating with the CGM 10 attached to the user and the insulin pump 20 or generating a signal for driving the insulin pump and the control unit 300 for determining an injection amount of the insulin pump, and checks once again the insulin injection amount determined by the reinforcement learning based on a threshold value, which is a safety threshold value, before the insulin injection amount determined by the reinforcement learning is changed to the final interface.

That is, the insulin injection amount determination system according to the present invention has four stacked structures based on reinforcement learning, thereby maximizing flexibility and adaptability.

In an exemplary embodiment of the present invention, the smart phone executes the algorithm according to the present invention, and accordingly, the interface layer of FIG. 1 performs a communication function between an external continuous glucose meter (CGM) 10, an insulin pump 20, and a controller to receive data or provide a signal for driving an external device.

In the system according to one embodiment of the present invention, several thresholds obtained on silica subjects are set, and to this end, the outer safety layer 200 is placed between the interface layer 100 and the control unit 300. This will be described in more detail below.

A control layer 300 based on deep reinforcement learning is provided on the outer safety layer 200.

In an embodiment of the present invention, the amount of insulin is calculated based on at least one of the observed GM (g) value, the insulin dose (insulin-on-board, iob) that means insulin still valid in the previous bolus, the CGM velocity (dg/dt or vg) and the CGM acceleration (d2g/dt2 or ag). That is, by combining variables obtainable from blood glucose data obtained from at least a plurality of continuous blood glucose systems, the rate of increase and decrease and acceleration of blood glucose can be predicted, and when at least one of the variables is used, all of them fall within the scope of the present invention.

At the top of the system structure according to the present invention, there is a personalized safety layer 400 for personal safety. In an embodiment of the present invention, the personalized safety layer 400 includes two sub-modules, namely, a OffSI module 410 and a OnSL module 420, which will be described in more detail below.

In the present invention, three new adjustable, time-dependent control variables are used to determine the safety of the automatic insulin injection prescription between users, which are a coefficient of day (SD), a coefficient of night (SN), and a maximum insulin dose (maximum IOB, IOBmax), which limits the output value of the insulin injection amount based on personal information associated with TDD.

The OffSI module, which is a lower module of the personalized safety layer 400 according to an embodiment of the present disclosure, determines a correlation between the total insulin injection amount (TDD, total daily dose) and the three variables.

The OnSL module, which is another lower module, performs a function of securing adaptability of the PersonAI algorithm according to the present invention by updating and adjusting the control variable. The OnSL module adaptively and finely adjusts the control variables to ensure the performance and stability of a control algorithm for long-term automatic insulin infusion (AID). For example, one of the functions of the OnSL module is to set a priority between variables in order to prevent hypoglycemia and increase a Time-In-Range (TIR) within a range of a blood glucose value every week.

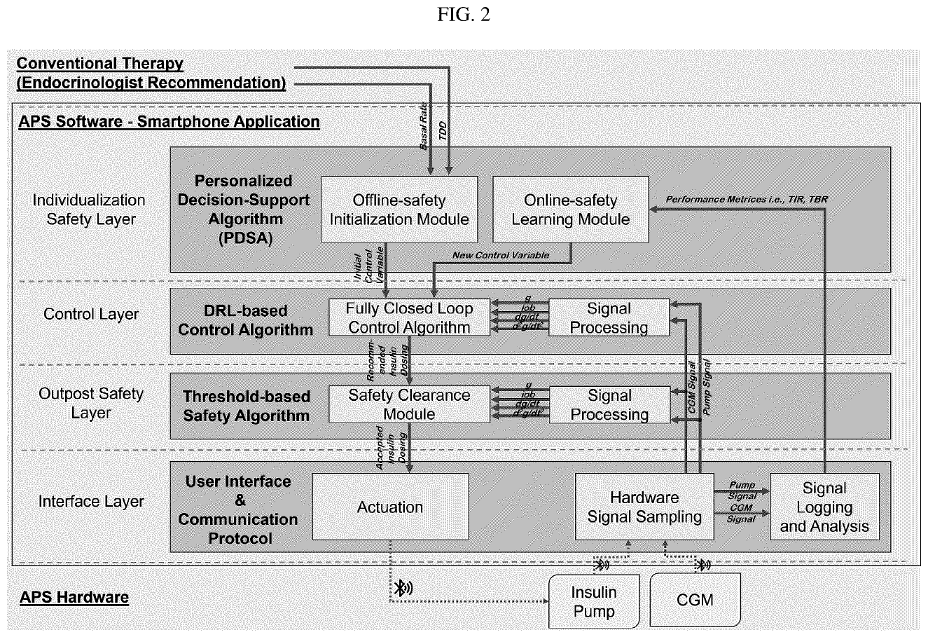

FIG. 2 is an operational schematic diagram of a system having a layered structure according to an embodiment of the present invention.

Referring to FIG. 2, in the present invention, two safety layers (the personalized safety layer 400 and the outer safety laser 100) are configured at both sides of the control layer 200, so that the insulin injection amount based on the actual individual-day information can be determined safely and elastically.

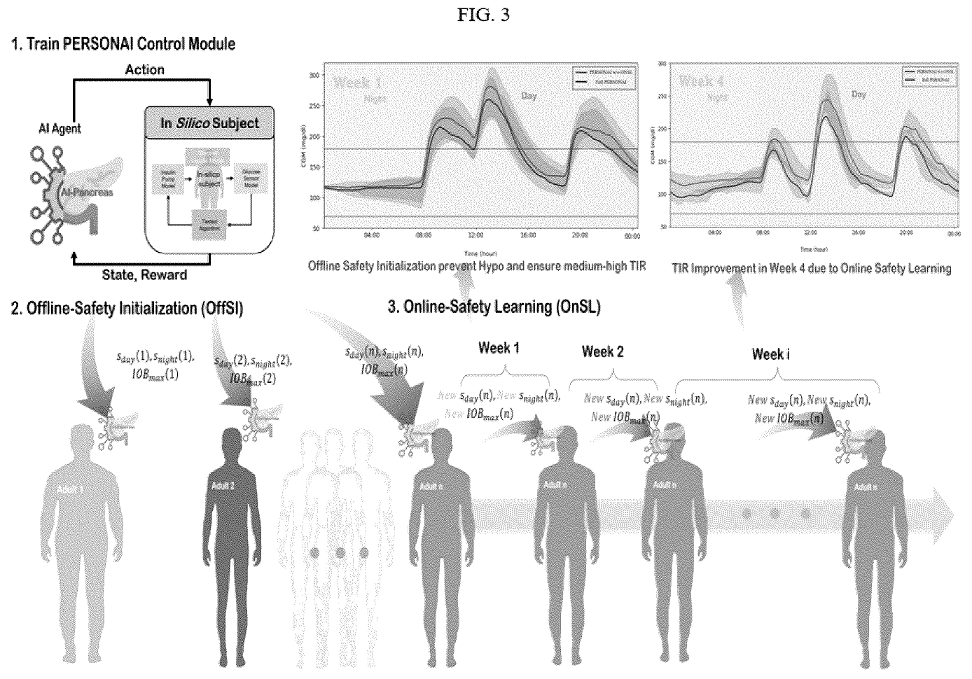

FIG. 3 is a conceptual diagram according to an embodiment of the present invention.

Referring to FIG. 3, blood glucose of a type 1 diabetes patient is controlled by using the personalized artificial intelligence-based automated insulin injection system according to the present invention, and in this case, the system according to the present invention is a fully closed loop scheme, and it is possible to predict blood glucose without a user's direct input of meal information.

Hereinafter, the present invention illustrated in FIGS. 1 to 3 will be described in more detail through Examples.

### Outpost Safety Layer-Safety Clearance Module

As described above, the system according to an embodiment of the present invention includes the separate outer safety layer 300 between the interface layer 100 and the control layer 200. The outer safety layer measures real-time metabolic state based on a continuous glucose monitor (CGM) and insulin infusion data, wherein g, vg, ag and iob values are used as input values. where g is the blood glucose level measured by the CGM, vg is the rate of change in blood glucose, ag is the rate of change in blood glucose, and iob is insulin-on-board, which is the amount of insulin already injected to lower current blood glucose. That is, g denotes a blood glucose change rate, ag denotes a variable which can be obtained from a blood glucose level, and all values are included in the scope of the present invention as long as at least one of g, vg, ag, and iob values is used as a variable.

As shown in FIGS. 1 and 2, information processed by the same signal processing module as input values of the personalized safety layer 400 and the control layer 200 is used for the outer safety layer. In the present invention, a clean g value is measured from the filtered CGM data, and vg and ag simply used values derived from g.

In order to obtain iob, which is another input value, the present invention calculated the total amount of insulin injected according to the following equation (1).

$${iob}_{t} = {\sum }_{k = 0}^{n - 1} {i}_{t - k} \cdot ( 1 - {F}_{k} ( SF ) ) \qquad ( 1 )$$

where iₜ₋ₖ is the insulin dose injected in the k time step before t, and Fₖ is the gamma cumulative density function (CDF) using SF as a scaling factor.

The present invention assumes that the cumulative iob remaining in the body of the user (e.g., 5 hours) operates using CDF. k is the number of time steps at which insulin dose data is collected, and n is the maximum number of time steps at which insulin dose data is collected during the time assuming that accumulated iob remains in the body of the user. For example, when it is assumed that an insulin operation is performed in the body for 5 hours and iob is collected at an interval of 5 minutes, n is 60.

In order to solve the problem of the prior art that the personalized iob is not suitable for fully automated insulin infusion in particular, the scale factor (SF) was used as described above.

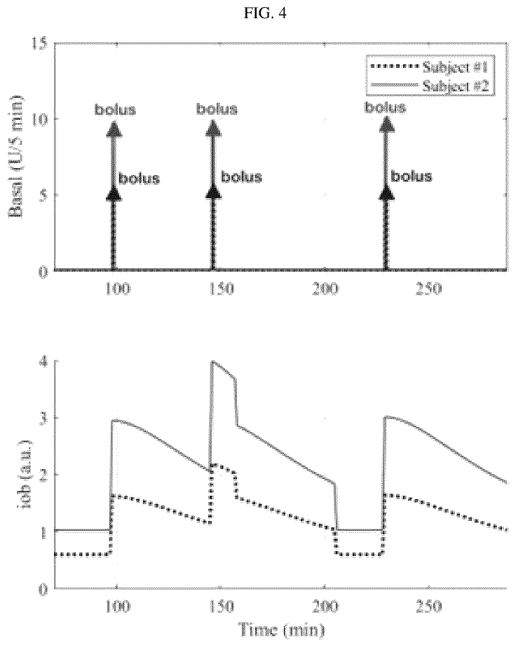

FIG. 4 is a diagram comparing generalized iob effects between two subjects with different TDD values.

Since the system according to the present invention is based on the Model-Free approach, an output safety layer is needed to improve reliability from problems in a serious external environment, such as CGM errors.

The present invention suppresses a Basal rate (CSII pump insulin dose (i)) by a hard thresholding method in which a coefficient whose absolute value is lower than a threshold value is set to 0, instead of suggesting a CSII (BR) according to the prior art.

In an embodiment of the present invention, three types of limit values are provided based on the time-dependent upper boundary values imax and iobmax of i and iob and the lower boundary value gmin of g.

First, two conditions for insulin limitation are as shown in Equation (2) below.

$$\hat{ι} ( t ) = \{ \begin{matrix}0 , & g ( t ) < {gs}_{min} or iob ( t ) > {iob}_{max} \\ \hat{ι} ( t ) , & other\end{matrix} \qquad ( 2 )$$

where î(t) is the insulin injection amount, which is the final output value of the control layer.

The allowable insulin injection according to the following one operation instruction can be defined as iₘₐₓ=BR [U/h], where the equation is as follows (3).

$$i ( t ) = \{ \begin{matrix}{i}_{max} , & i ( t ) > {i}_{max} \\ \hat{ι} ( t ) , & other\end{matrix} \qquad ( 3 )$$

Third, the possibility of a problem such as hypoglycemia should also be predicted, wherein the CSII pump insulin dose (i) is i(t)=0.

### Deep Reinforcement Learning (DRL)-Based Control Layer

As described above, the control layer of the system according to the present invention is based on a deep reinforcement learning (DRL) framework, and thus, in an embodiment of the present invention, the fully closed rope-automatic insulin injection problem is schematized with a Markov Decision Process consisting of six states where S is the user's hypoglycemic state (probabilistic observation value=0).

In the present invention, four values were used as input variables of the control layer, which are blood glucose g(t) (mg/dL), insulin dose (insulin-on-board, iob), CGM velocity (dg/dt or vg) and CGM acceleration (d2g/dt2 or ag).

The operation interval A is assumed to be continuous, and the amount of insulin injected by the CSII pump is defined as ī.

For learning, in the present invention, the operation interval A is set to 0<A<basalₘₐₓ. Here, basalₘₐₓ is the basal rate (BR) size allowed for the CSII pump to inject insulin (i.e., 30 U).

The state transfer constant P defined in the physiological model depends on each individual metabolic kinetics (i.e., UVA/Padova model and Hovorka model).

The compensation function R is related to the degree of blood glucose control, and this is a factor that reflects an optimization purpose using R: (sₜ, aₜ) and ν⊆[0.1] which is a discount factor that determines a trade-off between immediate compensation and delayed compensation.

In the present invention, two types of compensation functions were used, one being a long-term compensation function and the other being a short-term compensation function. Here, the long-term compensation function is to simulate basal insulin secretion of β cells, and the short-term compensation function is to simulate basal excess insulin secretion of β cells. Therefore, in the present invention, the discount factor is set to γ=0.99.

In one embodiment of the present invention, with respect to insulin prescription strategies, the Soft Actor Critic (SAC) algorithm was adopted to learn blood glucose control strategies (see T. Haarnoja, A. Zhou, K. Hartikainen, G. Tucker, S. Ha, J. Tan, et al., “Soft actor-critic algorithms and applications,” arXiv preprint arXiv vol. 1812, no. 05905, 2018; T. Haarnoja, A. Zhou, P. Abbeel, and S. Levine, “Soft actor-critic: Off-policy maximum entropy deep reinforcement learning with a stochastic actor,” International conference on machine learning, vol. 35, no. 80, pp. 1861-1870. PMLR, 2018.)

In an embodiment of the present invention, a policy action, which is an output value of the algorithm, corresponds to a desired amount of insulin injection at each preset time step (5 minutes in an embodiment of the present invention). The policy network in the present invention generates μ, log(σ) as an output value, which parameterizes a normal distribution N(μ,σ), and the operation is distributed according to a sigmoid normal distribution or

$$sigmoid ( z ) , z \sim 𝒩 ( μ , σ ) .$$

According to the SAC algorithm according to an embodiment of the present invention, the maximum entropy learning is corrected in a continuous operation region in which the probabilistic policy π is expressed as a parameter Ø.

According to an embodiment of the present invention, the maximum entropy objective function of the deep learning policy π_{Ø} may be represented by the following equation (4), wherein the equation is learned to maximize the objective function J(π).

$$J ( π ) = {\sum }_{t = 0}^{T} {E}_{( {s}_{t} , {a}_{t} ) \sim P ( {s}_{t - 1} , {π}_{ϕ} ( {s}_{t - 1} ) )} [ R ( {s}_{t} , {a}_{t} ) + α H ( {π}_{ϕ} ( . \vert {s}_{t} ) ) ] \qquad ( 4 )$$

where Σₜ E(sₜ,aₜ)˜P(sₜ₋₁, π_{ϕ}(sₜ₋₁))[R(sₜ,aₜ)] is the sum of the compensation (or cumulative compensation), H is entropy, and the relative importance to the compensation is determined by the temperature parameter.

In an embodiment of the present invention, an optimal temperature parameter was calculated using a conventional automatic temperature adjustment method. Each variable and parameter of the objective function is considered with reference to T. Haarnoja, A. Zhou, K. Hartikainen, G. Tucker, S. Ha, J. Tan, et al., “Soft actor-critic algorithms and applications,” arXiv preprint arXiv, vol. 1812, no. 05905, 2018.

In order to secure prescription safety between users corresponding to universal safety, not individual safety, the present invention sets a time-dependent insulin delivery limit value with respect to a calculation value of a DRL-based control layer. That is, in each step k, the limit value of the following equation (5) is applied to the calculated value of the control layer.

$$\hat{ι} := \{ \begin{matrix}\overline{ι} . {S}_{N} + π , & {τ}_{1} < {t}_{i} \leq {τ}_{2} \\ \overline{ι} . {S}_{D} + π , & otherwise\end{matrix} \qquad ( 5 )$$

where τ1=00:00 a.m. and τ2=07:00 a.m., wherein π is defined as BRmin/2, BRmin corresponds to BR/60 [U/min)], and ī an output value of the deep reinforcement learning according to the present invention.

In an embodiment of the present invention, the day's adjustment coefficient S_{D}>the night's adjustment coefficient S_{N}, S_{D} is determined by a calibration method described below, and S_{N} is defined by the following equation (6).

$${S}_{N} := \{ \begin{matrix}{b}_{1} {S}_{D} , & TDD \geq 50 \\ {b}_{2} {S}_{D} , & otherwise\end{matrix} \qquad ( 6 )$$

where b1 and b2 are the limit values of 0.3 and 0.2, respectively, and b1>b2.

Finally, in the present invention, iobₘₐₓ is defined as the following equation (7), which is included in the outer safety layer equation (2).

$${iob}_{max} := \{ \begin{matrix}2 , & TDD \geq 50 \\ 1.5 , & otherwise\end{matrix} \qquad ( 7 )$$

### Personal Safety Layer

The present invention provides a new PDSA algorithm for the personal safety layer, which will be described in more detail below.

### (1) Offline-Safe Start OffSI Module

The OffSI module according to the present invention provides an individual initial value for control variables of the artificial intelligence-based insulin injection system according to the present invention for all treatment patients.

As described above, the most realistic method for personalizing the control algorithm model without retraining is to subdivide the algorithm processing aggressiveness based on the ICR, BW, TDD, age, and gender of type 1 diabetic patients. This personal information is linked to the interindividual variability of insulin sensitivity (IS). Accordingly, the module according to the present invention is a OffSI module based on a regression model, which defines the association between TDD and optimized control variables.

In the present invention, an optimized control variable (i.e., SD) of each TID-VP (type 1 virtual diabetic) was found through a correction repetition procedure. On the other hand, other parameters, i.e., iobmax and SN, were determined from the hard threshold based on TDD, as shown in Equations (6) and (7), respectively.

In-silico testing experiments were performed according to protocols that specify the time and amount of meals.

A high SD value indicates an aggressive control value, which can be seen at higher infusion insulin values and may be suitable for some type 1 diabetics with higher TDD.

On the other hand, a low SD value represents a stable control value around the known value, which may be sufficient for some patients to maintain glucose in the normal range and to inhibit blood glucose elevation.

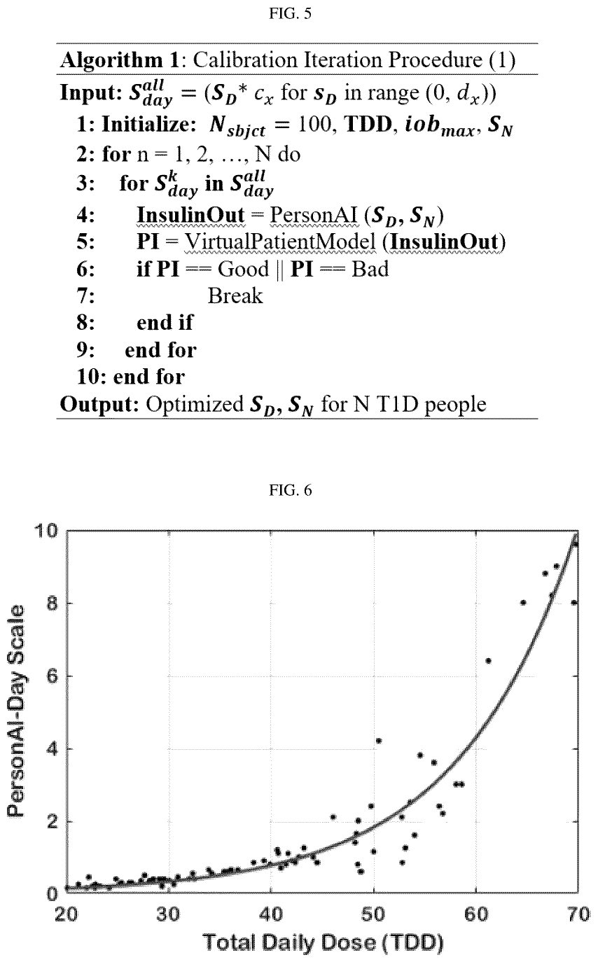

A calibration iteration procedure according to the present invention is illustrated in Algorithm 1 of FIG. 5. In this, it is cₓ∈[0.05, 0.1] and dₓ∈[2.5, 5] Therefore, the input value of the calibration repetition procedure according to the present invention is an SD set having an increment of 0.1 within a range of 0-5 with respect to TDD≥50, and an increment of 0.05 in TDD<50. In each iteration of Algorithm 1, the following performance index was calculated as an embodiment of the present invention.

$$PI = \frac{TIR}{1 - ( Β . TBR )} \qquad ( 8 )$$

Here, the TIR and the time below the range were calculated from glucose concentration time in the normal range (between 70 and 180 mg/dL per day), and glucose time in the hypoglycemic state (less than 70 mg/dL), respectively. β>1 is an adjustable parameter.

In each experimenter iteration, if certain conditions were met, a quick rest was performed.

For example, a “good” state is defined as a repetit ion TIR≥90% day for five consecutive s^{k}day, and a “bad” state is defined as a repetition TIR25% day for five consecutive s^{k}d ay.

Finally, the regression equation used in the present invention is shown in FIG. 6, and FIG. 6 is a graph showing the correlation between TDD and the control variable SD.

This may be represented by the following formula (9).

S_{d}Init=a·e^{b·TDD} (9)

Here, constants a and b were 0.013 and 0.086, respectively.

### OnSL Module

In an embodiment of the present invention, an object of the OnSL module is to update a control variable and improve a performance index in order to prevent a hypoglycemic accident.

Since the intra-day IS in the patient varies in each type 1 diabetic patient, the purpose of the OnSL module in one embodiment of the present invention is divided into daily and weekly, e.g., the daily pattern of insulin sensitivity (IS) is lower in the morning (morning) than at noon and night (lunch and dinner) on average, and other uncontrolled parameters (e.g., CGM) may fail. Specifically, in an embodiment of the present invention, the focus was on preventing hypoglycemia every day and increasing the TIR every week.

At each date k, the update rule has blood glucose of less than 60 mg/dL (TBR60), and follow performance based on performance percentage of TBR60ₓ={TBR60_{D}, TBR60_{N}, TBR60_{W}} (wherein D corresponds day, N corresponds night, W corresponds to TBR60).

TBR 60 was selected as the update algorithm according to an embodiment of the present invention in consideration of an error from a CGM value, and the update rule according to the present invention is as follows.

$${δ}_{( k + 1 )} = \{ \begin{matrix}δ ( - {f}_{1} ) & TBR {60}_{wk} > 0 and {S}_{D} \geq 0.15 \\ δ ( 1 - {f}_{2} ) & TBR {60}_{wk} > 0 and {S}_{D} < 0.15\end{matrix} \qquad ( 10 )$$

Here, δ={SD, SN} is a control variable of day and night updated by the algorithm according to the present invention. In an embodiment of the present invention, iobₘₐₓ is not updated daily in consideration of the stability of control.

The constraints f1 and f2 represent gains of 0.2 and 0.1, respectively.

A similar update rule was applied to weekly updates. In an embodiment of the present invention, a percentage constant was used to update the control variable. Thus, at each date j, the average TIRₓ={TIR_{D}, TIR_{N}, TIR_{W}} was calculated, and at date k the update rule follows the following equation (11).

$${δ}_{( k + 1 )} = \{ \begin{matrix}{S}_{{D}_{k}} ( 1 + {θ}_{1} ) , & \overline{{TIR}_{{W}_{k}}} \leq 0.7 \\ {S}_{{D}_{k}} ( 1 + {θ}_{2} ) , & otherwise\end{matrix} \qquad ( 11 )$$

Here, δ may be changed based on a specific performance matrix obtained daily, and constants θ1 and θ2 represent gains of 0.2 and 0.1. In one example of the present invention, when TIR_{W}>0.9, θ1>θ2, and the control variable was maintained. However, if any one of the conditions of TBR60_{W}>0 and TIR_{W}<80 at the subsequent date (k+1) is satisfied, the update process is resumed again. From Equation (10), it can be seen that the control parameter is updated if any one of the conditions is satisfied. Accordingly, any one or all of the δ variables may or may not be updated in one iteration process. The present invention further relates to maintaining the current variable value if a condition is not satisfied in a particular iteration (i.e., S_{DK}=S_{DK+1}).

### Experimental Example 1

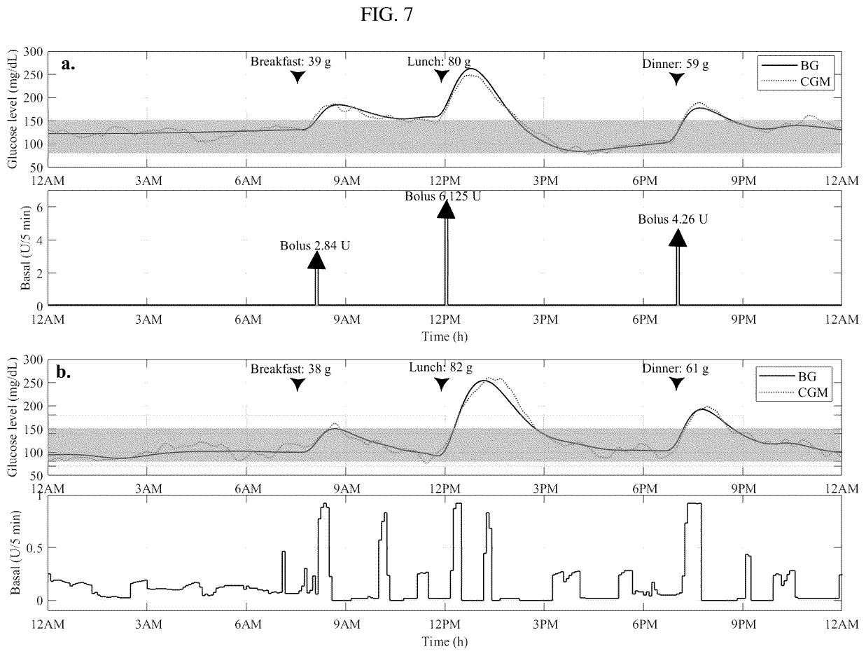

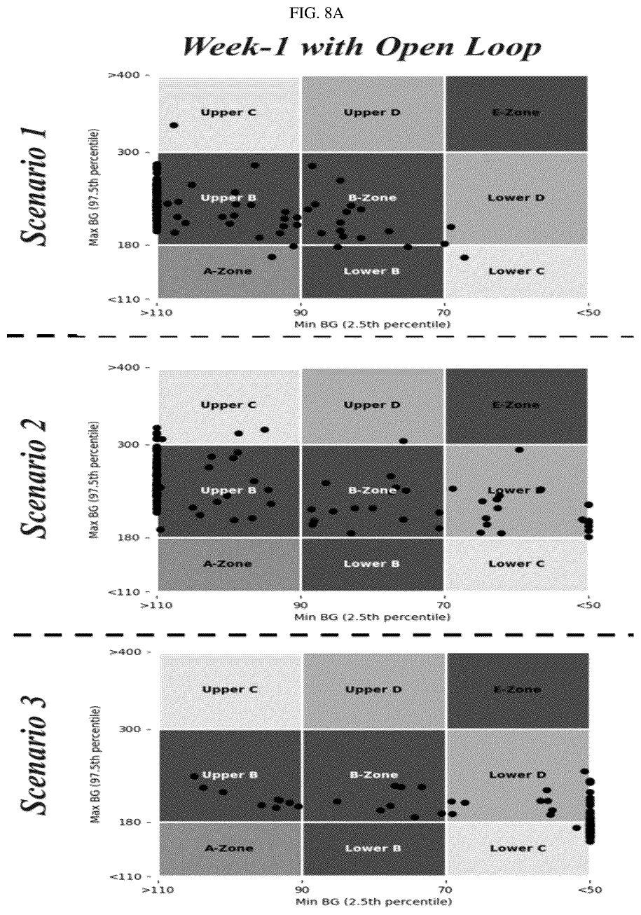

FIG. 7 is a graph comparing three CHOs having average contents of 40 g, 80 g, and 60 g for 1 day, and shows a result of comparing (a) an open loop (including a meal guide) using a conventional basal Bolus calculator with (b) a completely closed loop (without a meal guide) according to the present invention.

Referring to FIG. 7, it can be seen that the control method of the base-bolus method according to the related art and the control result of the micro-bolus-based method of the fully automated system according to the present invention, in which a high insulin bolus is required at each meal time, and it can be seen that when insulin is predicted in advance through the system according to the present invention, it is possible to perform insulin control at a level of the related art control method for inputting a meal amount or having superior performance.

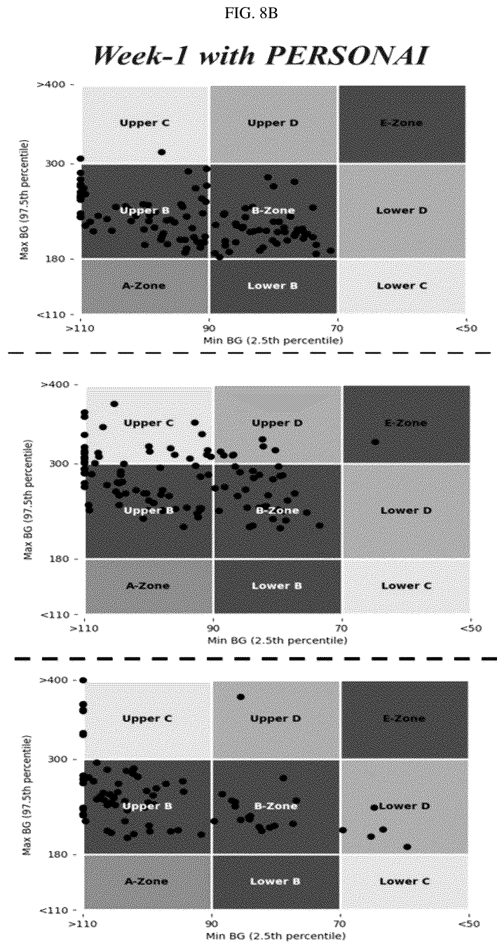

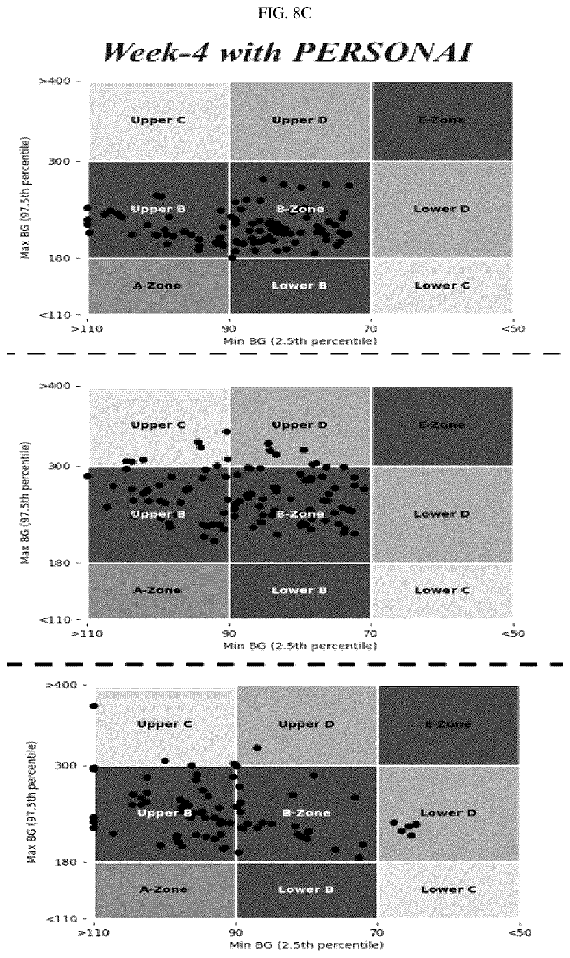

FIGS. 8A to 8C are the result of using the first two weeks of data obtained in the entered meal-open loop treatment and the last four weeks of data obtained in the fully closed loop of the present invention with unentered meal-personal AI.

In FIGS. 8A to 8C, in the case of scenario 1 and scenario 2, a total of 110 VPs (virtual patients) were used, whereas in scenario 3, only 10 VPs of UVA/Padova were used, and thus each point in the graph of scenario 3 was calculated based on CGM readings over 24 hours.

Here, in scenario 1, the Hovorka model of the UVA/Padova model and the built-in variability of daily IS embedded in DMMS.R were used. Each TID (type 1 diabetes) VP consumed a fixed amount of food at a specific intake time. In scenario 2, we tested an algorithm that consumed a relatively large amount of 90 g of carbohydrates in the morning, lunch, and evening. This scenario is closely mimicked the test protocol of Dassau et al. (2015). Earlier mentioned in Dassau et al. 2015, this scenario may exhibit a troublesome stress test for the AP controller, particularly for the FCL-AID. Finally, scenario 3 was used to determine the importance of the proposed OnSL module as a more challenging and realistic scenario (Gondhalekar et al., 2018). We tested the scenario designed in UVA/Padova TID VP here, but modified the original version and introduced additional daily variability in insulin uptake and IS. The nominal pattern of IS presented by Visentin et al. (2015) was implemented and randomly selected for each in silico subject. In addition, the daily variability in sensitivity was characterized by random ±50% variation in nominal IS from the beginning to the end of the test.

During the last 31 days of the simulated scenario and the proposed algorithm during the first 2 weeks of the open loop according to the prior art. It shows that the algorithm according to the present invention has succeeded in customizing and adapting to each patient state.

While the present disclosure has been described with reference to the embodiments illustrated in the figures, the embodiments are merely examples, and it will be understood by those skilled in the art that various changes in form and other embodiments equivalent thereto can be performed. Therefore, the technical scope of the disclosure is defined by the technical idea of the appended claims. The drawings and the forgoing description gave examples of the present invention. The scope of the present invention, however, is by no means limited by these specific examples. Numerous variations, whether explicitly given in the specification or not, such as differences in structure, dimension, and use of material, are possible. The scope of the invention is at least as broad as given by the following claims.

What is claimed is:

1. A personalized artificial intelligence-based insulin dose determination system by a computing terminal capable of communicating with an insulin pump and a continuous blood glucose system used by a user, the system comprising:
an interface layer capable of communicating with the insulin pump and the continuous blood glucose system, and signal-processing information from the insulin pump and the continuous blood glucose system;
a control layer receiving the signal-processed information from the interface layer and generating output information associated with an insulin infusion amount;
an outer safety layer determining whether or not the output information generated from the control layer satisfies a preset threshold condition, and delivering the output information to the interface layer when the output information satisfies the preset threshold condition; and
a personalized safety layer receiving prescription information including Total Daily Dose of Insulin (TDD) information of the user from the outside to determine a personalized safety control variable, and transmitting the determined control variable as an input variable of the control layer.

2. The personalized artificial intelligence-based insulin dose determination system of claim 1, wherein the control layer determines, as output information, the amount of insulin injected into the user using at least one of a continuous blood glucose level (g), an insulin-on-board (iob), a continuous blood glucose rate (dg/dt or vg), and a continuous blood glucose acceleration (d2g/dt2 or ag) measured by the continuous blood glucose system as input information from the signal-processed information.

3. The personalized artificial intelligence-based insulin dose determination system of claim 2, wherein the insulin-on-board iobt at time t is determined by the following equation:
$${iob}_{t} = {\sum }_{k = 0}^{n - 1} {i}_{t - k} \cdot ( 1 - {F}_{k} ( SF ) )$$
where iₜ₋ₖ is the insulin dose injected in the k time step before t, and Fₖ is the gamma cumulative density function (CDF) using SF as a scaling factor, k is the number of time steps at which insulin dose data is collected, and n is the maximum number of time steps at which insulin dose data is collected during the time assumed that accumulated iob remains in the body of the user.

4. The personalized artificial intelligence-based insulin dose determination system of claim 2, wherein the control layer determines the amount of insulin injected into the user as output information using a deep-reinforced learning model.

5. The personalized artificial intelligence-based insulin dose determination system of claim 4, wherein the deep-reinforced learning model uses a soft actor critical (SAC) algorithm, and the control layer determines a blood glucose control policy of the soft actor critical (SAC) algorithm at preset time intervals.

6. The personalized artificial intelligence-based insulin dose determination system of claim 5, wherein the control layer uses the soft actor critical (SAC) algorithm model for the purpose of maximizing an objective function J(π) according to the following equation:
$$J ( π ) = {\sum }_{t = 0}^{T} {E}_{( {s}_{t} , {a}_{t} ) \sim P ( {s}_{t - 1} , {π}_{ϕ} ( {s}_{t - 1} ) )} [ R ( {s}_{t} , {a}_{t} ) + α H ( {π}_{ϕ} ( . \vert {s}_{t} ) ) ]$$
where Σₜ E(sₜ,aₜ)˜P(sₜ₋₁,π_{ϕ}(sₜ₋₁))[R(sₜ,aₜ)] is the compensation sum, H is the entropy, and α is the temperature parameter.

7. The personalized artificial intelligence-based insulin dose determination system of claim 4, wherein the control layer determines the insulin injection amount as an output variable in any one or more of the following three limit value ranges:
Insulin infusion during the day (SD);
Insulin infusion during the night (SN); and
Maximum insulin-on-board value (iobmax).

8. The personalized artificial intelligence-based insulin dose determination system of claim 7, wherein the insulin dose is determined by the following equation:
$$\overline{i} := \{ \begin{matrix}\overline{i} . {S}_{N} + π , & {τ}_{1} < {t}_{i} \leq {τ}_{2} \\ \overline{i} . {S}_{D} + π , & otherwise\end{matrix}$$
where î is final output information of the control layer, t₁ wherein represents a time during the day, ī represents an insulin infusion amount initially determined using a deep-reinforced learning model, τ₁ and τ₂ represent a start time and an end time of a night time, SD represents a coefficient with respect to the day time, SN represents a coefficient with respect to the night time, π represents BRmin/2, BRmin represents BR/60 [U/min], and BR represents a basal insulin rate.

9. The personalized artificial intelligence-based insulin dose determination system of claim 8, wherein the insulin infusion amount (SN) during the night is a value obtained by multiplying the insulin infusion amount (SD) during the day by a coefficient between 0 and 1.

10. The personalized artificial intelligence-based insulin dose determination system of claim 9, wherein the coefficient is determined according to TDD (Total Daily Dose of Insulin).

11. The personalized artificial intelligence-based insulin dose determination system of claim 3, wherein the maximum insulin-on-board value (iobmax) is determined as a constant according to TDD (Total Daily Dose of Insulin), and the maximum insulin-on-board value (iobmax) is provided as a threshold of the outer safety layer.
